(19) United States
(12) Patent Application Publication
(10) Pub. No.: US 2016/0299137 A1
(43) Pub. Date: Oct. 13, 2016

(54) METHOD FOR DETECTING PATHOGENS USING MICROBEADS CONJUGATED TO BIORECOGNITION MOLECULES

(71) Applicant: FlO CORPORATION, Toronto (CA)

(72) Inventors: Michael Mordinson GREENBERG, Toronto (CA); Kevin Charles KAIN, Toronto (CA); Warren Che Wor CHAN, Toronto (CA)

(21) Appl. No.: 15/184,519

(22) Filed: Jun. 16, 2016

Related U.S. Application Data
(63) Continuation of application No. 12/279,639, filed on Jul. 28, 2009, now abandoned.

(30) Foreign Application Priority Data
Feb. 15, 2006 (CA) 2536698
Dec. 19, 2006 (CA) 2571904

(51) Int. Cl. G01N 33/569; G06F 19/00; G06F 17/30; B01L 3/00; H04W 4/02

(52) U.S. Cl. CPC G01N 33/569; B01L 3/502715; H04W 4/021; G06F 17/30241; G06F 19/345; B01L 2300/0627; B01L 2200/10; B01L 2300/02; B01L 2300/025

(57) ABSTRACT

A method and system are provided for the simultaneous detection and identification of multiple pathogens in a patient sample. The sample is combined with microbeads, which have been injected with quantum dots or fluorescent dye and conjugated to pathogen-specific biorecognition molecules, such as antibodies and oligonucleotides. Treatment options may be determined based on the identities of the pathogens detected in the sample.

### CROSS REFERENCE TO RELATED APPLICATIONS

This application is a continuation of Ser. No. 12/279,639 filed Jul. 28, 2009, which is a U.S. National Stage Application of International Application No. PCT/CA07/00211 filed Feb. 13, 2007, which claims priority from Canadian Patent Application No. 2,536,698 filed Feb. 15, 2006 and Canadian Patent Application No. 2,571,904, filed Dec. 19, 2006. The entireties of all the above-listed applications are incorporated herein by reference.

### FIELD OF THE INVENTION

The present invention relates to the field of detecting pathogens. In particular, it relates to a system and method for detecting, identifying, characterizing and surveilling pathogen and host markers, collecting and disseminating information concerning those pathogens and their hosts in real time to and from an instant location, providing instantaneous treatment recommendations and educational information.

### BACKGROUND OF THE INVENTION

Detection and characterization of an infectious disease is a complex process that ideally begins with the identification of the causative agent (pathogen). This has traditionally been accomplished by direct examination and culture of an appropriate clinical specimen. However, direct examination is limited by the number of organisms present and by the observer's ability to successfully recognize the pathogen. Similarly, in vitro culture of the etiologic agent depends on selection of appropriate culture media as well as on the microbe's fastidiousness. The utility of pathogen culture is further restricted by lengthy incubation periods and limited sensitivity, accuracy and specificity.

When in vitro culture remains a feasible option, the identification and differentiation of microorganisms has principally relied on microbial morphology and growth variables which, in some cases, are sufficient for strain characterization (i.e. isoenzyme profiles, antibiotic susceptibility profiles, and chematographic analysis of fatty acids).

If culture is difficult, or specimens are not collected at the appropriate time, the detection of infection is often made retrospectively, if at all, by demonstrating a serum antibody response in the infected host. Antigen and antibody detection methods have relied on developments in direct (DFA) and indirect (IFA) immunofluorescence analysis and enzyme immunoassay (EIA)-based techniques, but these methods can also possess limited sensitivity.

These existing methods have several drawbacks. First, the process can take several days to return results. In the case of highly communicable and/or dangerous pathogens, confirmation of pathogen type may not be received until the host has already exposed others or has passed beyond treatment. Second, the transportation of samples to laboratories for culture growth increases the risk of errors, such as misidentifying the sample, or exposure of unprotected personnel to a sample containing a highly communicable pathogen. Thirdly, the pathogen tests are limited based on the suspected pathogen list provided by the observer (i.e. doctor), meaning that additional unsuspected pathogens are not tested for but may be present.

Related to this method of diagnosis is the response to an outbreak of infectious disease. If an outbreak is suspected or detected, the existing response is the hundreds of years old method of quarantine. In cases of infectious disease outbreaks for which appropriate treatments and/or sensitive, specific, and rapid screening/diagnostic tests are lacking, quarantine remains the only means of preventing the uncontrolled spread of disease. When infection is suspected simply based on epidemiological grounds, or even based on comparable disease presentation, healthy or unexposed individuals may be quarantined along with infected individuals, elevating their likelihood of contracting the disease as a consequence of quarantine. Availability of a rapid confirmatory test for the pathogen in question would greatly reduce the time spent in quarantine, and would therefore reduce the likelihood of contacting the disease from truly infected persons.

Although quarantine remains a method of last resort for protecting public health, delays in providing a correct diagnosis, and subsequently appropriate treatment, occur on a daily basis in hospitals and physician's offices alike. The problem stems from the fact that many diseases have very similar clinical presentations in the early stages of infection, and in the absence of a thorough patient/travel history, malaria or SARS for example, can be misdiagnosed as the common flu (i.e. fever, chills), albeit with potentially fatal consequences. Had a multi-pathogen test which differentiates diseases with similar presentations been available, a tragedy may have been averted.

In contrast to reliance on morphological characteristics, pathogen genotypic and proteomic traits generally provide reliable and quantifiable information for the detection and characterization of infectious agents. Moreover, microbial DNA/RNA can be extracted directly from clinical specimens without the need for purification or isolation of the agent.

On a global scale, molecular techniques can be applied in a high throughput manner in screening and surveillance studies monitoring disease prevalence and distribution, evaluation of control measures, and identification of outbreaks.

Point-of-care diagnostic devices (PDDs) have been developed for a number of individual infectious diseases. In most cases these assays are immunochromatographic single colorimetric strip tests designed to detect a single infectious agent (either a pathogen-specific antigen or an antibody response to one) in a small volume of blood or serum.

None of these current assays has the capability to detect multiple pathogens or simultaneously detect genomic and proteomic markers of multiple pathogens. Similar limitations exist for other rapid diagnostic assays. Since almost all these tests rely on a single visual colorimetric change for their readout, the opportunities to detect multiple pathogens are severely impeded and the majority of current PDDs are restricted to the detection of a single pathogen. Consequently, evaluating patients for potential infectious agents or testing a unit of blood for common transmissible agents requires multiple consecutive point-of-care tests to be performed, complicating clinical management, slowing results and significantly escalating costs.

Many PDDs do not meet what are considered essential requirements including: ease of performance, a requirement for minimal training, the generation of unambiguous results, high sensitivity and specificity, the generation of same day results (preferably within minutes), relative low cost, and no requirement for refrigeration or specialized additional equipment.

In summary, despite current availability of excellent diagnostic reagents (e.g. antibody and nucleic acid probes) that recognize specific targets for many microbial pathogens, the current strategies have inadequate performance characteristics. Contributing to this is the fact that these reagents are conjugated to organic dyes, gold-labelled particles or enzymes that lack sufficient sensitivity to be detected at the single molecule level. Furthermore, the current PDD platforms and detection schemes typically rely on single macroscopic colorimetric changes and are not well suited to the simultaneous detection of multiple pathogens.

More recent advances in molecular diagnostics, including real-time PCR combined with automated specimen processing, have addressed a number of the limitations of earlier “in-house” and non-standardized gene amplification assays. These assays represent a significant advance in detecting, quantifying, and characterizing many microbes and currently represent the “gold” or reference standard for infectious disease diagnostics for a number of pathogens. However, these assays are still complex, expensive, and require specialized equipment, creating a number of barriers to their potential application at point-of-care.

Finally, current genomic or proteomic detection strategies require a sample processing and technical commitment to one strategy or the other. There is no current capacity to simultaneously detect both antigenic targets for some pathogens and genetic targets for others. This limits the simultaneous detection of preferred pathogen-specific targets and presents a barrier to fully exploiting the complementary power of both strategies.

A system is needed which enables pathogen detection, identification and characterization, as well as host characterization in a much more timely manner than existing methods. Preferably, such a system would support a modular pathogen selection platform, based on the specific needs of the caring physician or clinic in the context in which the device is used (i.e. for screening or diagnosis). Further, the system would also enable simultaneous detection, identification and characterization of multiple pathogens in a single sample whereby the pathogens are differentiated by optical pathogen-specific profiles stored in a pre-existing database.

### SUMMARY OF THE INVENTION

According to an aspect of the invention there is provided a method of performing one or more of: detecting, identifying and characterizing pathogens and characterizing pathogen hosts using markers for pathogens and hosts, comprising the steps of: a) preparing a marker-detection medium containing signatures of the identity and characteristics of pathogens and optionally of hosts; b) collecting a sample from a host; c) combining the sample with the marker-detection medium and d) analyzing the signatures to detect, identify and characterize the pathogens, and optionally, characterize the host.

Preferably, the sample collected is a blood sample, although plasma, serum, cerebral spinal fluid (CSF), bronchioalveolar lavage (BAL), nasopharyngeal (NP) swab, NP aspirate, sputum and other types of samples can also be used, and the marker detection system is a pathogen-detection medium preferably comprising microbeads conjugated to biorecognition molecules (BRMs) and the microbeads are injected with quantum dots or a similar fluorescent particle or compound. Also preferably, each of the microbeads contains a unique combination of quantum dots to provide a unique optical barcode associated with each microbead for detecting unique pathogen-specific and/or host-specific signatures.

Preferably, the analysis step comprises illuminating the microbead-pathogen sample with a laser as it flows through a microfluidic channel and collecting the resulting spectra with a spectrophotometer/CCD camera, photomultiplier tube and/or a collection of avalanche photodetectors (APDs). Each spectrum correlates with a previously assigned pathogen.

Optionally, the method may include producing a list of host characterization markers associated with said host sample as part of analysis step d).

Optionally, the method may include an additional step e) of providing a list of treatment options based on the list of pathogens generated in analysis step d).

Optionally, the method may also include step f) of correlating geographic location information data with the list of pathogen and host markers generated in analysis step d) via a GPS locator.

Preferably, the method further includes an additional step g) of transmitting, preferably wirelessly, said list of pathogen markers and said list of host identifier markers and said geographic location data to a remote database as well as transmitting treatment and educational information from the database to the filed device. It will be appreciated that the steps of the process are not necessarily conducted in the specified order.

The method further includes detection of pathogen-conjugated microbeads in a flow stream propelled by electrokinetic or hydrodynamic flow through a microfluidic channel. As the barcoded beads pass a laser beam at one end of the channel, the spectra emitted by the quantum dots within the beads, (as part of the barcode), or outside the beads (as part of a bead-pathogen complex detection mechanism, which may include fluorophores as described below) are collected by a spectrometer/CCD camera system, photomultiplier tube and/or a collection of APDs and analyzed by appropriate software.

According to the invention, there is disclosed a method of detecting one or more pathogens, identifying one or more pathogens, characterizing one or more pathogens and/or characterizing a pathogen host. The method is for use with a clinical sample collected from a host that potentially contains one or more target molecules. The method includes a detection medium providing step, a detection complex forming step, a spectral reference database providing step, and an analysis step. In the detection medium providing step, a detection medium is provided which contains pathogen-specific/host marker identification complexes for respective detection of pathogens and host markers. The pathogen-specific/host marker identification complexes preferably include microbeads conjugated to respective pathogen-specific/host marker biorecognition molecules (BRMs). Each of the microbeads preferably contains quantum dots, fluorescent dyes, or combinations thereof, such that each of the microbeads is adapted to emit one or more spectra as a first signal. In the detection complex forming step, the clinical sample is combined with the detection medium and the detection molecules. Both the pathogen-specific/host marker identification complexes and the detection molecules are adapted to bind with the target molecules if present in the clinical sample, to generate detection complexes. Each of the detection molecules is further adapted to emit one or more spectra as a second signal. In the spectral reference database providing step, a spectral reference database of pathogen-specific/host marker reference spectra is provided. In the analysis step, the detection complexes are flowed, under influence of flow forces, preferably through a microfluidic channel and preferably through a laser beam, such that resulting spectral signals are emitted from different types of the detection complexes. The resulting spectral signals include the first signal, the second signal, or a combination thereof. In the analysis step, the resulting spectral signals are analyzed with a detection element in a handheld diagnostic device by: (a) detecting the resulting spectral signals; (b) collecting and translating the resulting spectral signals, into a translated optical code for each of the different types of detection complexes, preferably using solid state photodetectors of the detection element which are adapted to emit electrons in direct response to the resulting spectral signals; and (c) matching each aforesaid translated optical code with a corresponding one of the pathogen-specific/host marker specific spectra in the spectral reference database to produce a list of pathogens contained within the clinical sample, and a list of pathogen/host characteristics.

According to an aspect of one preferred embodiment of the invention, the method may preferably be for use with a blood sample, a plasma sample, CSF (Cerebrospinal Fluid), a serum sample, BAL (Bronchoalveolar lavage), NP (nasopharyngeal) swabs, NP aspirates, and/or sputum as the clinical sample.

According to an aspect of one preferred embodiment of the invention, the solid state photodetectors may preferably include a collection of Avalanche Photodetectors.

According to an aspect of one preferred embodiment of the invention, the collection of Avalanche Photodetectors may preferably be arranged in series.

According to an aspect of one preferred embodiment of the invention, each of the microbeads may preferably contain a unique combination of the quantum dots, preferably based on color and/or intensity of the quantum dots, and preferably for emission of a unique spectrum as the first signal for each of the pathogen-specific/host marker identification complexes.

According to an aspect of one preferred embodiment of the invention, the detection complexes may preferably identify the pathogen/host characteristics, preferably by the resulting spectral signals, and preferably in the form of the combination of the first signal and the second signal emitted by the detection molecules.

According to an aspect of one preferred embodiment of the invention, at least one of the detection molecules may preferably include a fluorophore, preferably to emit the second signal.

According to an aspect of one preferred embodiment of the invention, the fluorophore may preferably be conjugated to an anti-human IgG molecule, an anti-human IgM molecule, an anti-pathogen/host marker detection antibody, and/or an oligonucleotide sequence.

According to an aspect of one preferred embodiment of the invention, in the analysis step, analysis of the resulting spectral signals may preferably be additionally performed by: a combined spectrophotometer/CCD (Charge-coupled Device) system, a photomultiplier tube, or a combination thereof.

According to an aspect of one preferred embodiment of the invention, the microfluidic channel may preferably include a PDMS (polydimethylsiloxane) cast channel which is, preferably, plasma treated and/or bound to a glass slide.

According to an aspect of one preferred embodiment of the invention, the flow forces may preferably be electrokinetic and/or hydrodynamic forces.

According to an aspect of one preferred embodiment of the invention, the spectral reference database may preferably be located on-board the diagnostic device.

According to an aspect of one preferred embodiment of the invention, the method may preferably also include a geographic location collection step of collecting geographic location data, preferably from the diagnostic device, and preferably for at least one of the pathogens and/or the host.

According to an aspect of one preferred embodiment of the invention, the geographic location data may preferably be collected via a GPS-enabled (Global Positioning System) element that is, preferably, within the diagnostic device.

According to an aspect of one preferred embodiment of the invention, the method may preferably also include a geographic location determining step, a remote database providing step, a transmission step, and/or a reception step. In the geographic location determining step, geographic location data is preferably determined for the diagnostic device and, preferably, for at least one of the pathogens and/or the host. In the remote database providing step, a remote database is provided, preferably at a location that is geographically remote from the diagnostic device. In the transmission step, the list of pathogens contained within the clinical sample, the list of pathogen/host characteristics, and/or the geographic location data is wirelessly transmitted, preferably, to the remote database. In the reception step, the list of pathogens contained within the clinical sample, the list of pathogen/host characteristics, and/or the geographic location data, for each aforesaid transmission step of each aforesaid diagnostic device is preferably received, collated and/or stored, preferably in the remote database.

According to an aspect of one preferred embodiment of the invention, the method may preferably also include an additional step of providing a list of treatment options, preferably based on the list of pathogens contained within the clinical sample.

According to an aspect of one preferred embodiment of the invention, the detection medium may preferably contain at least three aforesaid identification complexes, each preferably for detection of a different one of the pathogens and/or the host markers.

According to an aspect of one preferred embodiment of the invention, the identification complexes may preferably be for detection of HIV, Hepatitis B and/or Hepatitis C.

According to an aspect of one preferred embodiment of the invention, the identification complexes may preferably be for detection of HIV, Hepatitis B, Hepatitis C, malaria and/or Dengue virus.

According to another aspect of the invention a system of components is provided which is capable of executing any of the above methods.

According to the invention, therefore, there is additionally disclosed a system for detecting pathogens, identifying pathogens, characterizing pathogens and/or characterizing pathogen hosts. The system is for use with a clinical sample collected from a host that potentially contains one or more target molecules. The system is also for use with detection molecules adapted to bind with the target molecules if present in the clinical sample and emit one or more spectra as a second signal. The system includes a detection medium, a handheld diagnostic device, and a spectral reference database of pathogen-specific/host marker reference spectra. The detection medium contains pathogen-specific/host marker identification complexes for respective detection of pathogens and host markers. The pathogen-specific/host marker identification complexes preferably include microbeads conjugated to respective pathogen-specific/host marker biorecognition molecules (BRMs). Each of the microbeads preferably contains quantum dots, fluorescent dyes, or combinations thereof, such that each of the microbeads is adapted to emit one or more spectra as a first signal. The detection medium is operative to be combined with the clinical sample and with the detection molecules. The pathogen-specific/host marker identification complexes are adapted to bind with the target molecules if present in the clinical sample, such that the pathogen-specific/host marker identification complexes, the detection molecules, and the target molecules form detection complexes. The handheld diagnostic device preferably includes a microfluidic platform and a detection element. The microfluidic platform is operative to drive the detection complexes, using flow forces, preferably through a laser-illuminated region in a microfluidic channel, such that resulting spectral signals are emitted from different types of the detection complexes. The resulting spectral signals include the first signal, the second signal, or a combination thereof. The detection element is operative to detect the resulting spectral signals. The detection element preferably has solid state photodetectors adapted to collect and translate the resulting spectral signals, by emission of electrons in direct response to the resulting spectral signals, into a translated optical code for each of the different types of detection complexes. The spectral reference database is operative to match each aforesaid translated optical code with a corresponding one of the pathogen-specific/host marker specific spectra in the spectral reference database, to generate a list of pathogens contained within the clinical sample, and a list of pathogen/host characteristics.

According to an aspect of one preferred embodiment of the invention, at least one of the microbeads may preferably contain quantum dots to provide the first signal. The system may preferably be for use with a blood sample, a plasma sample, CSF (Cerebrospinal Fluid), a serum sample, a BAL (Bronchoalveolar lavage), a NP (nasopharyngeal) swab, a NP aspirate, and/or a sputum sample as the clinical sample.

According to an aspect of one preferred embodiment of the invention, each of the microbeads may preferably contain a unique combination of the quantum dots, preferably for emission of a unique spectrum as the first signal for each of said pathogen-specific/host marker identification complexes.

According to an aspect of one preferred embodiment of the invention, the system may preferably be for use with a signal generating molecule, preferably as a constituent of at least one of the detection molecules. The signal generating molecule may preferably operatively emit the second signal.

According to an aspect of one preferred embodiment of the invention, the system may preferably be for use with a fluorophore, preferably as the signal generating molecule.

According to an aspect of one preferred embodiment of the invention, the system may preferably be for use with an anti-human IgG molecule, an anti-human IgM molecule, an anti-pathogen/host marker detection antibody, and/or an oligonucleotide sequence, preferably conjugated to the fluorophore.

According to an aspect of one preferred embodiment of the invention, the solid state photodetectors may preferably include a collection of Avalanche Photodetectors.

According to an aspect of one preferred embodiment of the invention, the collection of Avalanche Photodetectors may preferably be arranged in series.

According to an aspect of one preferred embodiment of the invention, the detection element may preferably include a spectrometer/CCD (Charge-coupled Device) system, a photomultiplier tube, or a combination thereof, preferably for additional analysis of the resulting spectral signals.

According to an aspect of one preferred embodiment of the invention, the diagnostic device may preferably be operative to display a list of treatment options, preferably based on the list of pathogens generated.

According to an aspect of one preferred embodiment of the invention, the system may preferably also include a laser, preferably operative to illuminate the laser-illuminated region in the microfluidic channel.

According to an aspect of one preferred embodiment of the invention, the microfluidic channel may preferably include a PDMS (polydimethylsiloxane) cast channel which is, preferably, plasma treated and/or bound to a glass slide.

According to an aspect of one preferred embodiment of the invention, the flow forces may preferably be electrokinetic and/or hydrodynamic forces.

According to an aspect of one preferred embodiment of the invention, the detection element may preferably include a filter. Preferably, the filter is operative to direct the resulting spectral signals to the solid state photodetectors, to a spectrometer, to a photomultiplier tube, and/or to a combination thereof.

According to an aspect of one preferred embodiment of the invention, the spectral reference database may preferably be on-board the diagnostic device.

According to an aspect of one preferred embodiment of the invention, the system may preferably also include a remote database and/or a connection, preferably on the diagnostic device and/or to enable communication with the remote database. Preferably, the remote database contains data concerning different pathogens and/or data concerning pathogen/host characteristics.

According to an aspect of one preferred embodiment of the invention, the connection may preferably be provided by a wireless communications network.

According to an aspect of one preferred embodiment of the invention, the connection may preferably include a transmission element, preferably operative to transmit the list of pathogens and/or the list of pathogen/host characteristics, preferably to the remote database.

According to an aspect of one preferred embodiment of the invention, the transmitter may preferably be operative to automatically initiate transmission to the remote database, preferably upon generation of the list of pathogens and/or the list of pathogen/host characteristics.

According to an aspect of one preferred embodiment of the invention, the diagnostic device may preferably also include a GPS (Global Positioning System) locator element, preferably to provide geographic location data, preferably associated with the clinical sample.

According to an aspect of one preferred embodiment of the invention, the system may preferably also include a locator element, a remote database, a wireless transmission element, and a wireless reception element. The locator element may preferably be operative to determine geographic location data, preferably for the diagnostic device and, preferably, for at least one of the pathogens and/or the host. The remote database may preferably be provided at a location geographically remote from the diagnostic device. The wireless transmission element may preferably be operative to wirelessly transmit, preferably to the remote database, the data concerning pathogens contained within the clinical sample, the data concerning pathogen/host characteristics, and/or the geographic location data. The wireless reception element may preferably be operative to receive, collate and/or store, preferably in the remote database, the data concerning pathogens contained within the clinical sample, the data concerning pathogen/host characteristics, and/or the geographic location data, preferably for each wireless transmission, preferably from each aforesaid diagnostic device.

According to an aspect of one preferred embodiment of the invention, the locator element may preferably include a GPS (Global Positioning System) locator element, preferably to determine the geographic location data.

According to an aspect of one preferred embodiment of the invention, the identification complexes may preferably be provided as one or more lyophilized powders.

According to an aspect of one preferred embodiment of the invention, the BRMs may preferably include native, recombinant and/or synthetic pathogen and/or host specific antibodies and/or antigens and/or oligonucleotides complementary to pathogen and/or host genes of interest, or a combination thereof.

According to an aspect of one preferred embodiment of the invention, the detection medium may preferably contain at least three aforesaid identification complexes, each preferably for detection of a different one of the pathogens and/or the host markers.

According to an aspect of one preferred embodiment of the invention, the identification complexes may preferably be for detection of HIV, Hepatitis B and/or Hepatitis C.

According to an aspect of one preferred embodiment of the invention, the identification complexes may preferably be for detection of HIV, Hepatitis B, Hepatitis C, malaria and/or Dengue virus.

According to an aspect of one preferred embodiment of the invention, the system may preferably be for use with a lyophilized powder, preferably as at least one of the detection molecules.

The advantages of the present invention include a vast reduction in the amount of time necessary to identify pathogens in a patient sample, compared with most methods currently in use, as well as the ability to provide rapid on-site information concerning treatment and quarantine measures for any identified pathogens. Another advantage is the ability to collect patient and pathogen data in a global database and mine the information contained in this database to produce trends and tracking measures for various pathogens and their hosts, which information may be used for surveillance, research, therapeutic design, and other purposes.

Other and further advantages and features of the invention will be apparent to those skilled in the art from the following detailed description thereof, taken in conjunction with the accompanying drawings.

### BRIEF DESCRIPTION OF THE DRAWINGS

The invention will now be described in more detail, by way of example only, with reference to the accompanying drawings, in which like numbers refer to like elements, wherein:

FIG. 1 is a flow chart detailing the series of steps in the inventive method disclosed herein;

FIG. 2 is a block diagram for a pathogen detection device; and

FIG. 3 is a block diagram of multiple devices communicating with a central database.

### DETAILED DESCRIPTION OF THE PREFERRED EMBODIMENTS

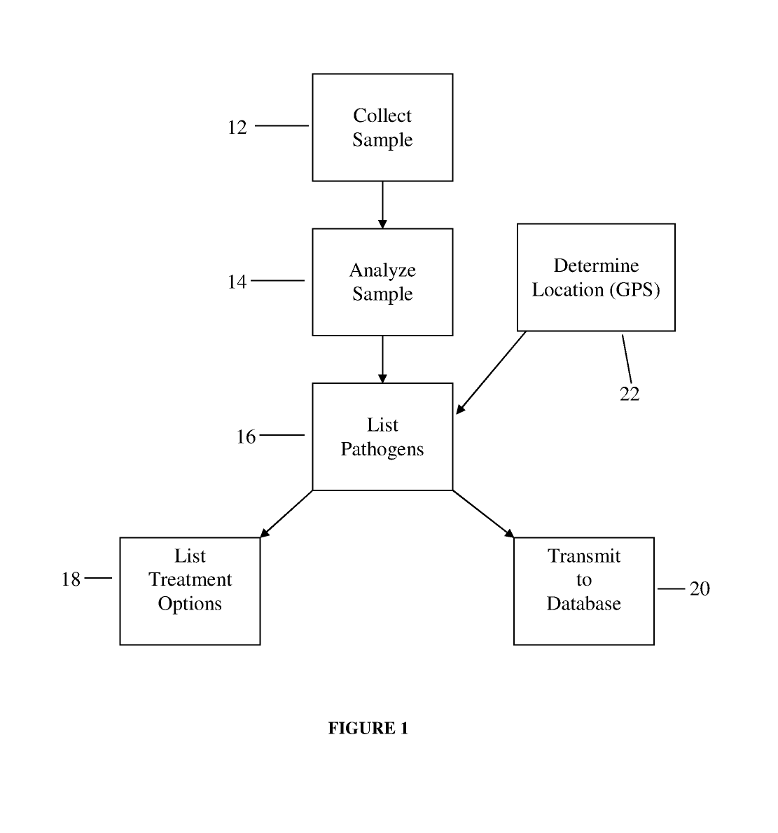

Referring now to FIG. 1, the present inventive method is described by a series of steps set out in a flowchart.

The first step 12 is to collect a sample from a host (e.g. a human, animal or environmental sample), preferably a blood sample, although plasma samples, serum samples, CSF, BAL, NP aspirates, NP swabs, sputum and other types of physical samples can be used, as appropriate. This sample is then analyzed 14 and a list of pathogens identified in the sample is generated 16. A GPS receiver 22 determines the location of the sample reader and thus, the sample. The list of identified pathogens and the location information are both sent 20 to a central database for storage and processing. Meanwhile, a list of treatment options is displayed at 18, based on the identified pathogens, for the operator's consideration.

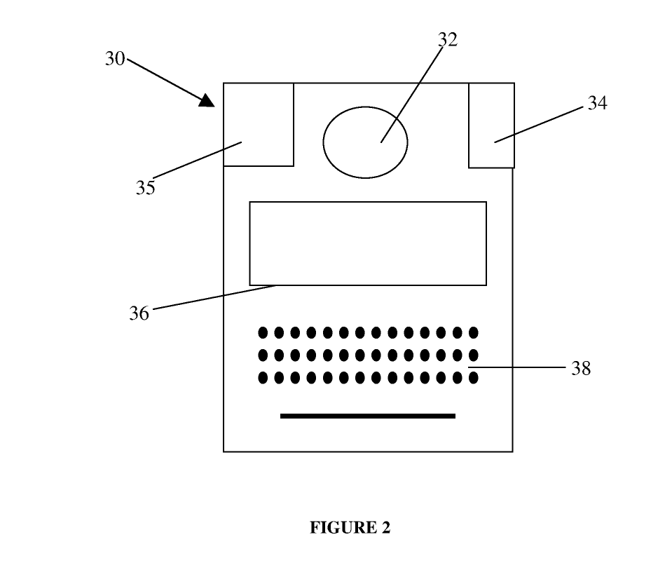

The analysis 14 is performed by a pathogen detection device 30 as shown in FIG. 2. This device 30 is portable, preferably hand-held, and has an outlet 32 for receiving a sample and a display 36 to show the list of detected pathogens within the sample. An input device 38, such as a keyboard, is also provided to enable scrolling and viewing of the display and input of additional information (field notes, etc.). Pathogens in a sample are identified based on matching of spectra to previously stored data corresponding to each pathogen supported by the device. The spectra database may be an internal database on the device 30 (kept in flash memory or similar storage to allow for updating) or retrieved by communicating with an external database. A GPS receiver 35 is also preferably located in the device 30, along with a display showing the GPS co-ordinates. Ideally, all communication is conducted wirelessly for maximum range and portability. The pathogen detection device 30 is ideally capable of detecting multiple pathogen, multiple BRMs from the same pathogen as well as host markers within a single sample, and preferably markers of different types, such as protein-based markers and gene-based markers.

The method of detection used can be varied among suitable available methods, however, a preferred method is the use of biorecognition molecules (BRMs) conjugated to quantum dot-doped microbeads or nanobeads/nanoparticles. Alternatives include single quantum dots or fluorophores conjugated to BRMs. Quantum dots, also known as semiconductor nanocrystals, are electromagnetically active nanotechnology-based particles, ranging in size from 2 nanometers (nm) to 8 nm. A particularly useful property of quantum dots is that they are fluorescent, that is they emit light after brief illumination by a laser. In addition, quantum dots of different sizes will fluoresce in different colors and the fluorescing color can be modified by the particle's shape, size and composition. BRMs are biological molecules that bind only to a single other biological molecule and are pathogen specific. For example, “antibodies” are BRMs that bind to proteins and “oligonucleotide probes” are BRMs that bind to complementary gene sequences (e.g. DNA or RNA). Pathogens and hosts have both unique and shared genetic and protein markers, and each marker can be bonded to by a specific BRM.

A microbead, which is a polystyrene (or similar polymer) bead that can be 100 nanometers-10 micrometers in diameter and doped with a collection of quantum dots, is physically conjugated to a BRM. By introducing unique combinations of quantum dots of different sizes (i.e., colors) and at different concentrations into the microbeads, microbeads with thousands of distinctive combinations of quantum dot colors and intensities can be created. When a laser illuminates the microbeads, the quantum dots fluoresce to produce a distinctive combination of colors. These color combinations are an example of a barcode, in this case an optical bar code, analogous to a UPC symbol, and similar known types of imprinted barcodes. Since each BRM recognizes a distinct pathogen or host marker and each microbead has a unique barcode, each BRM-conjugated microbead provides a barcode for the specific pathogen or host marker recognized by its BRM. These BRM-conjugated microbeads, as well as BRM-conjugated quantum dots, may be lyophilized into a powder and provided in the sample analysis kit.

To differentiate between BRM-conjugated beads bound to pathogens and those that are not, an additional confirmatory detection signal in the form of anti-human IgG, and/or an anti-human IgM molecule, or a pathogen-specific antibody (i.e. anti-X antibody), or an oligonucleotide (complementary to a pathogen gene of interest) conjugated to a fluorophore, is included. The readout of a successful pathogen detection test comprises the bead barcode signal and a second signal generated by the fluorophore.

One example of pathogen detection is an antigen capture system. The antigen capture system includes a capture antibody (i.e. a BRM) which is bound to the barcoded microbead which is responsible for capturing the antigen from the sample. A second antibody (detection antibody) which recognizes the pathogen antigen/protein then binds to the complex. This detection antibody is conjugated to a fluorophore. When the sample is analyzed, if the signal for the detection antibody is not detected, the pathogen does not register as detected, either because it is not present in the sample or because of assay failure. The latter case is eliminated if the correct signals from the positive control sample, i.e. detection of the appropriate bar code of the BRM-quantum dot-containing microbead run in parallel with all clinical tests are detected.

Another example of pathogen detection is an antibody capture system. In the antibody capture system the BRM which is bound to the barcoded microbead is a pathogen-specific antigen or protein (natural, recombinant, or synthetic). The complementary antibody to the antigen, if present in the clinical sample would bind the antigen attached to the bead. This complex is recognized by the addition of a secondary (detection) anti-human antibody (Anti-Human IgM or Anti-Human IgG). This detection antibody is conjugated to a fluorophore. Again, when the sample is analyzed, if the signal for the detection antibody is not detected alongside the signal from the bead barcode the pathogen does not register as detected, either because it is not present in the sample, or due to assay failure. The latter case is eliminated if the expected signals from positive control sample, as mentioned above, register correctly.

Still another example of pathogen detection is a genomic analysis system. In the genomic analysis system the BRM which is bound to the barcoded microbead is a pathogen-specific oligonucleotide (RNA or DNA) (1-25 bases in length). Upon addition to the sample, the oligonucleotide will hybridize to its complementary sequence on the pathogen gene. A second oligonucleotide sequence complimentary to a downstream portion of the gene of interest is subsequently added and will hybridize to the gene, if present. This second sequence is conjugated to a fluorophore. Again, when the sample is analyzed, if the signal for the second sequence is not detected, the pathogen does not register as detected, either because it is not present in the sample or because of assay failure. A correctly detected positive control sample as referred to above eliminates the latter scenario.

The biological (e.g. blood) sample is added to a vial, and different pathogen markers bind the various microbeads carrying specific pathogen BRMs. The combined sample is then washed or otherwise treated to remove extraneous matter and unattached microbeads. The detection antibodies conjugated to the fluorophores are then added to produce a bead-sample-detector complex.

The bead-sample-secondary detector complex is flowed through a microfluidic channel via hydrodynamically or electrokinetically-driven flow and passed through a laser beam located at one end of the channel. The laser beam illuminates the quantum dots in the complex and the emitted wavelengths are guided to either a spectrometer/CCD system, photomultiplier tube and/or a series of APDs. Signal deconvolution software translates the signal and the corresponding optical code is compared to pathogen-specific spectra stored in the database of pathogens or host characteristics supported by the detection device. Then, a list of detected pathogens and pathogen and host characteristics is produced. The response time from the taking of the original biological sample to the production of the pathogen list can be measured in minutes.

Ideally, the pathogen detection device 30 is a portable, hand-held device with an integrated laser and spectrophotometer, photomultiplier tube and/or series of APD units, specifically designed PDMS microfluidic channel chips, a supply of BRM conjugated barcoded beads for identification of various pathogens as well as appropriate bead-pathogen complex detection markers (quantum dot, fluorophore, small bead labeled IgG/IgM/anti-pathogen antibodies or oligonucleotides). The device 30 may store a pathogen identity database on board, or access a remote database, preferably via the Internet, preferably wirelessly, and identify the pathogen from a remote, central database. If an on-board database is used, a communications system 34 for contacting and receiving updates from a larger, central database is provided.

The pathogen detection device 30 may include a GPS tracking device which transmits specific geographic information, preferably wirelessly to the same central database.

Once the pathogen list is produced, the pathogen detection device 30 may additionally provide further information of value to the diagnosing doctor. Ideally, a treatment protocol is provided (step 18), including any special measures necessary to avoid communication of the pathogen. Other information, such as pathophysiology, disease history and bibliographic references can be provided, enabling the pathogen detection device 30 also to be used as an educational tool in the appropriate scenarios.

An outbreak scenario for use of the device in a standard pathogen detection setting follows. An airport is a point of entry representing a major pathogen travel vector, as well as presenting problems with implementing traditional detection and quarantine methods. By equipping medical staff with a number of pathogen detection devices as described herein, and a supply of microbead sample vials able to detect pathogens typically transmitted by travelers, incoming passengers can be processed on-site by taking a blood sample and injecting it into a sample vial. The analysis is performed by the pathogen detection device within minutes and the sampled passenger can be quickly released or redirected for treatment and observation, as necessary. While a single device is limited in processing capability, the ability to provide multiples of identical devices can enable processing of passengers in a matter of hours, not days. Faster processing allows appropriate treatment and quarantine measures to be taken earlier, and be more effective, reducing the probability of the pathogen spreading unchecked.

As an example, a pathogen detection device may contain BRM-conjugated barcoded microbeads for detection of three different pathogens, say, HIV, Hepatitis B and Hepatitis C. The microbeads associated with each pathogen have a separately identifiable barcode, for example, HIV may have red beads (e.g. detecting the antibody gp41 as indicator of HIV infection), Hepatitis B yellow beads (e.g. detecting the antibody NSP₄ as indicator of Hepatitis B infection), and Hepatitis C red-yellow beads (e.g. detecting the antibody anti-NSP₄ as indicator of Hepatitis C infection), and preferably all using orange probes-pathogen complex detection markers or any color-probe that is spectrally different than the color of the barcodes. Thus, the detection system can readily identify any detected pathogen merely by the wavelength (which identifies color) or intensity of the bead spectra.

From this model, the system can readily be expanded, for example, to five pathogens, adding, for example, pathogen detection microbeads for malaria and dengue virus. From there, extrapolation to more pathogens (10, 20, 100) is mostly limited by the ability to create a sufficient number of barcodes, which is based primarily on the doping of the microbeads and limits of the detection mechanism. As the number increases, barcodes may be based on intensity levels, as well as wavelength.

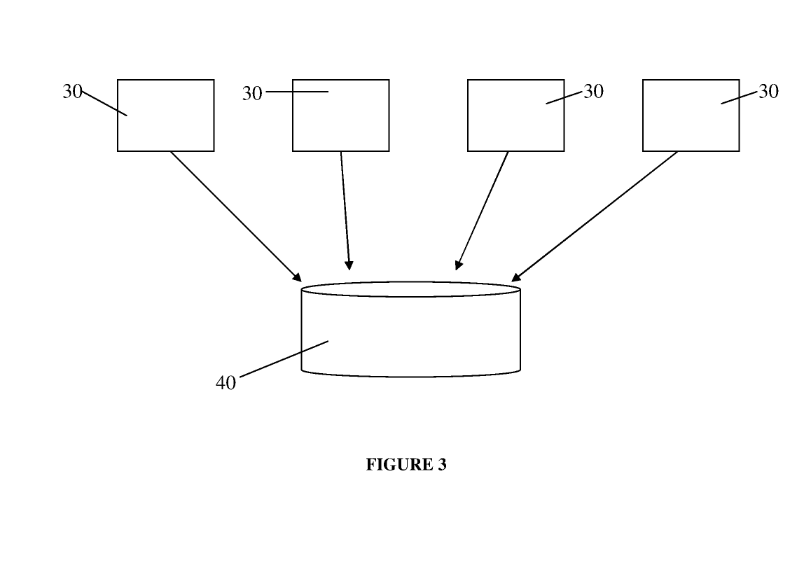

Detecting and providing a treatment protocol for a pathogen represents merely the first step in a potentially much larger process for tracking and controlling the spread of pathogens as shown in FIG. 3. Tailoring the device to be modular and be able to detect either an array of pathogens (i.e. BRMs for multiple pathogens) with similar clinical presentations, act as a screening tool (e.g. for identifying individuals vaccinated for selected diseases) or allowing physicians or clinics to select the pathogens of interest in their particular communities, allows for unprecedented diagnostic flexibility at the bedside. Incorporation of multiple BRMs for the same pathogen enhances detection accuracy and overcomes the limitations associated with use of single BRMs for pathogen detection (i.e. mutations and strain differences which may result in false negative or false positive results). The test results data along with the geographic location data (but no other information about the patient e.g. name, address and other privacy-protected data) provided by the GPS unit, are transmitted to a central database 40. The information is preferably sent wirelessly, and immediately upon generation of the pathogen list (step 20). The central database 40 is in contact with a substantial number of pathogen detection devices 30 at any given time.

The central database 40 can be local, national or global, or a combination of different databases of these types. Ideally, one top-level central database 40 is provided which receives information constantly from all devices 30 worldwide. Over time, the database becomes a repository of information on every pathogen supported by the detection platform lending itself to mining for, among others, frequency and global patterns of detection of pathogens, long-term pathogen trends (i.e. colonization of new territories), and correlations between pathogens and host markers which may indicate enhanced susceptibility or resistance to the disease.

What is claimed is:

1. A system for detecting pathogens, identifying pathogens, characterizing pathogens or characterizing pathogen hosts, said system being for use with a clinical sample collected from a host that potentially contains one or more target molecules, and being for use with detection molecules adapted to bind with said target molecules if present in the clinical sample and emit one or more spectra as a second signal, said system comprising:
a) a detection medium containing pathogen-specific/host marker identification complexes for respective detection of pathogens and host markers, wherein said pathogen-specific/host marker identification complexes comprise microbeads conjugated to respective pathogen-specific/host marker biorecognition molecules (BRMs), and wherein each of said microbeads contains quantum dots, fluorescent dyes, or combinations thereof, such that each of said microbeads is adapted to emit one or more spectra as a first signal, with said detection medium operative to be combined with the clinical sample and with said detection molecules, with said pathogen-specific/host marker identification complexes adapted to bind with said target molecules if present in the clinical sample, such that said pathogen-specific/host marker identification complexes, said detection molecules, and said target molecules form detection complexes;
wherein each of said microbeads contains a unique combination of said quantum dots for emission of a unique spectrum as said first signal for each of said pathogen-specific/host marker identification complexes;
b) a handheld diagnostic device, including:
i) a microfluidic platform that is operative to drive the detection complexes, using flow forces, through a laser-illuminated region in a microfluidic channel, such that resulting spectral signals are emitted from different types of said detection complexes, with said resulting spectral signals including said first signal, said second signal, or a combination thereof; and
ii) a detection element that is operative to detect said resulting spectral signals, and having solid state photodetectors adapted to collect and translate said resulting spectral signals, by emission of electrons in direct response to said resulting spectral signals, into a translated optical code for each of said different types of detection complexes; wherein said solid state photodetectors comprise a collection of Avalanche Photodetectors;
c) a spectral reference database of pathogen-specific/host marker reference spectra operative to match each said translated optical code with a corresponding one of said pathogen-specific/host marker specific spectra in said spectral reference database to generate a list of pathogens contained within the clinical sample, and a list of pathogen/host characteristics; and
a remote database containing data concerning different pathogens and data concerning pathogen/host characteristics, and a connection on said diagnostic device to enable communication with said remote database; and
wherein said diagnostic device is operative to display a list of treatment options based on the list of pathogens generated.

2. The system of claim 1, wherein at least one of said microbeads contains quantum dots to provide said first signal, and wherein said system is for use with a blood sample, a plasma sample, CSF (Cerebrospinal Fluid), a serum sample, a BAL (Bronchoalveolar lavage), a NP (nasopharyngeal) swab, a NP aspirate, or a sputum sample as the clinical sample.

3. The system of claim 1, for use with a signal generating molecule as a constituent of at least one of said detection molecules, with said signal generating molecule operatively emitting said second signal.

4. The system of claim 3, wherein said signal generating molecule is a fluorophore.

5. The system of claim 4, wherein an anti-human IgG molecule, an anti-human IgM molecule, an anti-pathogen/host marker detection antibody, or an oligonucleotide sequence, is conjugated to said fluorophore.

6. The system of claim 1, wherein said collection of Avalanche Photodetectors is arranged in series.

7. The system of claim 1, wherein said detection element comprises a spectrometer/CCD (Charge-coupled Device) system, a photomultiplier tube, or a combination thereof, for additional analysis of said resulting spectral signals.

8. The system of claim 1, further including a laser operative to illuminate said laser-illuminated region in said microfluidic channel.

9. The system of claim 1, wherein said microfluidic channel comprises a PDMS (polydimethylsiloxane) cast channel which is plasma treated, and bound to a glass slide.

10. The system of claim 1, wherein said flow forces are either electrokinetic or hydrodynamic forces.

11. The system of claim 1, wherein said detection element includes a filter operative to direct said resulting spectral signals to said solid state photodetectors, to a spectrometer, to a photomultiplier tube, or to a combination thereof.

12. The system of claim 1, wherein said spectral reference database is on-board the diagnostic device.

13. The system of claim 1, wherein said connection to said remote database is provided by a wireless communications network.

14. The system of claim 1, wherein said connection comprises a transmission element operative to transmit said list of pathogens and/or said list of pathogen/host characteristics to said remote database.

15. The system of claim 14, wherein said transmitter is operative to automatically initiate transmission to said remote database upon generation of said list of pathogens and/or said list of pathogen/host characteristics.

16. The system of claim 1, wherein the diagnostic device further comprises a GPS (Global Positioning System) locator element to provide geographic location data associated with said clinical sample.

17. The system of claim 1, further comprising a locator element operative to determine geographic location data for said diagnostic device and for at least one of said pathogens and said host;
wherein said connection on said diagnostic device comprises a wireless transmission element operative to wirelessly transmit, to said remote database, said data concerning pathogens contained within said clinical sample, said data concerning pathogen/host characteristics, and said geographic location data; and
further comprising a wireless reception element operative to receive, collate and store, in said remote database, said data concerning pathogens contained within said clinical sample, together with said data concerning pathogen/host characteristics, and together with said geographic location data, for each wireless transmission from each said diagnostic device.

18. The system of claim 17, wherein said locator element comprises a GPS (Global Positioning System) locator element to determine said geographic location data.

19. The system of claim 1, wherein said identification complexes are provided as one or more lyophilized powders.

20. The system of claim 1, wherein said BRMs comprise native, recombinant or synthetic pathogen and host specific antibodies or antigens or oligonucleotides complementary to pathogen or host genes of interest, or a combination thereof.
